(19) United States
(12) Patent Application Publication
(10) Pub. No.: US 2025/0277720 A1
(43) Pub. Date: Sep. 4, 2025

(54) IONIZING AN AEROSOL FOR ANALYSIS OF PARTICLES IN THE AEROSOL BY CHARGE DETECTION MASS SPECTROMETRY OR ION MOBILITY SPECTROMETRY

(71) Applicant: THE REGENTS OF THE UNIVERSITY OF CALIFORNIA, Oakland, CA (US)

(72) Inventors: Evan R. Williams, Oakland, CA (US); Conner C. Harper, Albany, CA (US)

(73) Assignee: THE REGENTS OF THE UNIVERSITY OF CALIFORNIA, Oakland, CA (US)

(21) Appl. No.: 19/193,890

(22) Filed: Apr. 29, 2025

Related U.S. Application Data
(63) Continuation of application No. PCT/US2023/081341, filed on Nov. 28, 2023.
(60) Provisional application No. 63/385,141, filed on Nov. 28, 2022.

(51) Int. Cl. G01N 1/22; G01N 1/40; G01N 27/62; H01J 49/16

(52) U.S. Cl. CPC G01N 1/2202; G01N 1/4022; G01N 27/62; H01J 49/165; G01N 2001/4027

(57) ABSTRACT

Devices and methods for ionizing an aerosol for analysis of particles in the aerosol. In one variant, the aerosol is intersecting with charged droplets formed by electrospray ionization (ESI) to ionize the aerosol. In another variant, the aerosol is passed through a plasma source to ionize the aerosol. In a further variant, the aerosol is passed through an ultraviolet (UV) ionizing source to ionize the aerosol. In all variants, the ionized aerosol is dried to yield charged, gas-phase analytes. The source of the aerosol can be a person's breath or a nebulizer. The aerosol can be dried in the inlet of a charge detection mass spectrometer (CDMS) or an inlet of an ion mobility instrument.

### CROSS-REFERENCE TO RELATED APPLICATIONS

This application claims priority to, and is a 35 U.S.C. § 111 (a) continuation of, PCT international application number PCT/US2023/081341 filed on Nov. 28, 2023, incorporated herein by reference in its entirety, which claims priority to, and the benefit of, U.S. provisional patent application Ser. No. 63/385,141 filed on Nov. 28, 2022, incorporated herein by reference in its entirety. Priority is claimed to each of the foregoing applications.

The above-referenced PCT international application was published as PCT International Publication No. WO 2024/118601 A1 on Jun. 6, 2024, which publication is incorporated herein by reference in its entirety.

### STATEMENT REGARDING FEDERALLY SPONSORED RESEARCH OR DEVELOPMENT

This invention was made with government support under GM139338 awarded by the National Institutes of Health. The government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to methods and devices for aerosol formation and ionization and more particularly to devices and methods for electrospray ionization (ESI), extractive electrospray ionization (EESI), plasma-based ionization, and ultraviolet (UV) photoionization of volatile and nonvolatile compounds, including proteins, large biomolecular assemblies, particles, and intact cells, that are (>100 kDa) contained in aerosol droplets or aerosol condensate. The apparatus enables the ionization of large molecules, molecular assemblies, and particles (>100 kilodaltons) directly from aerosol droplets that have diameters ranging from 0.1 μm to 100 μm and may efficiently route these molecules to the inlet of a charge detection mass spectrometer, ion mobility spectrometer, or other detection instrumentation for size and/or mass measurement.

### 2. Background Discussion

The devastating societal and economic impact of COVID-19 pandemic has made clear the need for a fast, untargeted viral screening tool that can identify individuals who are highly contagious in real-time. The continued loss of life, widespread closures of businesses and public venues, and countless other adversities all stem from the danger and uncertainty associated with the transmissibility of the previously unknown SARS-CoV-2 virus. The reverse transcription quantitative polymerase chain reaction (RT-qPCR) method is the “gold standard” in determining if an individual is infected with a virus such as SARS-CoV-2. However, qPCR-based tests do not quantify contagiousness, but merely an approximate copy number for a specific viral genome in the sampled nasopharyngeal fluid. Generally, these qPCR-based methods are slow, typically requiring 45-90 minutes with results that are often reported several days after sampling. Faster antibody-based methods that can be home-administered have been developed, but still require 15-30 minutes to administer.

Critically, because both qPCR and antibody-based testing methods are specific to a particular virus, they can take many months to develop and manufacture in meaningful quantities after an outbreak is detected, hampering efforts to control the initial spread of the disease. Both methods also have a significant cost associated with non-reusable materials and reagents and can be challenging and expensive to scale up for high-throughput screening.

Mass spectrometry (MS) and ion mobility spectrometry are powerful methods for biomolecule characterization. Gas-phase biomolecular ions can be directly formed from solution using electrospray ionization (ESI) and analyzed using a variety of MS and/or ion mobility techniques. Even when successful mass measurements are made for MDa-sized analytes, dynamic range suffers as a consequence of the increased heterogeneity because broadened peaks obscure low abundance components. Large particles or collections of particles (such as whole cells) can be weighed using sensitive surface acoustic wave sensors (SAWS) with a lower limit of few picograms, corresponding to a few teradaltons (TDa). However, this leaves a significant gap from ˜10 MDa to ˜1 TDa that is inaccessible using currently available commercial instrumentation capable of accurate mass measurements.

Accordingly, there is a need for detection devices that can make a quantitative measure of viral load using ionized aerosol from as little as a single breath (1-3 seconds) so that the extent of an individual's contagiousness could be determined with high throughput.

### BRIEF SUMMARY

Devices and methods of ionizing an aerosol droplet or aerosol condensate for analysis of particles contained in the aerosol droplet are provided. The ionization of MDa-sized analytes from aerosols has not previously been documented. Extractive electrospray ionization (EESI) is a technique to ionize both volatile and nonvolatile compounds, including proteins, in aerosols, but no EESI source designs expressly intended for the ionization of large biomolecules (>100 kDa) are believed to exist. Methods and apparatus for aerosol sampling and condensation are commercially available, but no designs expressly coupled to an ionization source to facilitate analysis of large biomolecules (>100 kDa) in the condensate are believed to exist.

Generally, the methods produce or provide a flow of an aerosol containing analytes of interest from a source such as a nebulizer or from the breath of a subject. The aerosol is then ionized or is condensed and then the condensate is subsequently ionized. Embodiments of the devices and methods preferably use one of several different ionization methodologies, including electrospray ionization, extractive electrospray ionization, plasma-based ionization generated with DC, AC, RF, microwave, or pulsed capacitor discharge, and UV photoionization. The ionized aerosol or condensate is then dried to yield charged, gas-phase analytes that are suitable for analysis by a charge detection mass spectrometer or an ion mobility spectrometer, for example.

The methods and devices enable the ionization of large molecules, molecular assemblies and particles (>100 kDa) directly from aerosol droplets with that have diameters ranging from 0.1 μm to 100 μm or directly from aerosol condensate to produce charged gas phase analytes for analysis. For example, the ionized molecules may be directly introduced to the inlet of a charge detection mass spectrometer or an ion mobility spectrometer for size and/or mass selection measurement.

The sensitivity achievable using these methods for analyzing aerosol contents allows measurement of statistically significant distributions of large biomolecules contained in the relatively dilute aerosols. This enables studies of aerosol droplet content, most notably the aerosols generated by human breath. Whole pathogen particles can be ionized and then weighed from these breath aerosols to distinguish between various viruses and bacteria that are relevant to human health. This also allows fast screening and characterization of the transmission risk for these aerosol-borne pathogens by directly measuring how infectious a person is from real time measurements of their breath.

For example, the device could be a component of a charge detection-based instrument or ion mobility instrument designed to quantitatively measure the number of virus and bacteria particles in human breath and provide information about the type of pathogen based on size and/or shape measurements. Such an instrument could screen for, quantify, and differentiate the presence of various pathogens in human breath, including aerosol-borne viruses, such as influenza, SARS-CoV-2, and rhinoviruses (common cold) and even aerosol-borne bacteria such as tuberculosis.

In combination with state-of-the-art charge detection mass spectrometry or ion mobility analysis, a quantitative measure of the viral pathogen load in as little as a single breath (1-3 seconds) could be obtained, such that the extent of an individual's contagiousness could be determined with high throughput. In combination with charge detection mass spectrometry, the measurement would be based on measuring pathogen molecular mass and is not targeted to a specific pathogen allowing for simultaneous screening for a broad spectrum of aerosol-borne diseases, including emergent viruses that are not yet well-characterized. Deployment of instruments based on this technology in key locations, such as airports, hospitals, and public venues, could significantly reduce the spread of disease thereby reducing negative impacts of future pandemics.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a functional block diagram of a method for ionizing aerosols and producing and delivering charged, gas-phase analytes for analysis according to one embodiment of the technology.

FIG. 2 is a schematic diagram of an apparatus with EESI aerosol ionization according to one embodiment of the technology. A flow of aerosols generated either by breath or nebulizer is intersected with charged droplets formed by ESI to produce ionized aerosols. The aerosols are then dried in the inlet of the CDMS or ion mobility instrument to form gaseous ions for analysis.

FIG. 3 is a schematic diagram of an apparatus with cold plasma aerosol ionization according to an alternative embodiment of the technology. A flow of aerosols is generated either by breath or nebulizer. The aerosols pass through tubing wrapped in coils connected to a high voltage RF power supply, which produces a cold plasma inside of the tubing. Aerosols are bombarded by the ionized molecules of the plasma, resulting in ionization of the aerosols. The aerosol size distribution can be measured either before or after this ionization. After ionization, the aerosols are then fully dried of solvent in the inlet of the CDMS or ion mobility instrument to form gaseous ions for analysis.

FIG. 4 is a schematic diagram of an apparatus with UV photoionization ionization according to an alternative embodiment of the technology. A flow of aerosols generated either by breath or nebulizer and contained in a UV transparent tubing is intersected with the beam of a UV diode laser. Aerosol particles can be ionized directly or indirection through photoionization of other gaseous species resulting in proton or other charge transfer to the aerosol particles. This results in charged aerosols that are then dried in the inlet of the CDMS or ion mobility instrument for mass and/or collision cross section analysis. Aerosol size can be measured both before and after photoionization. UV light can be generated using lasers, flash lamps, arc discharge sources or other methods of generating intense UV light.

FIG. 5 is a schematic diagram of an apparatus with a condenser that condenses sampled aerosols prior to ionization according to an alternative embodiment of the technology.

FIG. 6 is a mass spectrum plot generated from a solution of 1 μM myoglobin nebulized from pure water and directed into the ESI spray. The charge states observed (8+ and 9+) are indicative of a native protein conformation and the high abundance of holo-myoglobin (˜95%) relative to apo-myoglobin (˜5%) indicates that non-covalent complexes of relatively large biomolecules can be preserved when using EESI.

FIG. 7 is a mass histogram (bin size=3 MDa) plot generated from solution of nebulized from aqueous suspension of amidine-functionalized nanospheres (0.08% wt) and directed into an ESI spray of 0.5% aqueous acetic acid. The polydisperse nature of the nanospheres leads to a broad range of masses ranging from ˜100-400 MDa and centered at ˜220 MDa. The subpopulation at masses <100 MDa may be due to sample contamination or breakdown of larger species.

### DETAILED DESCRIPTION

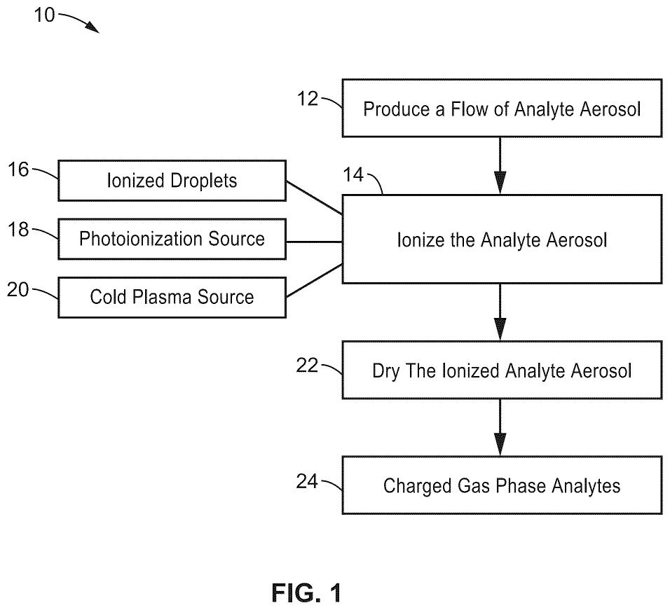

Referring more specifically to the drawings, for illustrative purposes, methods for ionizing an aerosol for analysis of particles in the aerosol are generally shown. Several embodiments of the technology are described generally in FIG. 1 to FIG. 7 to illustrate the characteristics and functionality of the devices, systems, materials and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, one embodiment of a method 10 for ionizing aerosols to yield charged, gas-phase analytes for analysis is shown schematically. The first step of method 10, shown at block 12, is to provide a flow or volume of aerosol from a source such as a nebulizer or similar source. The aerosol at block 12 may also come from the breath of a subject. The aerosol contains one or more types of analytes of interest such as aerosol-borne pathogens, large molecules, molecular assemblies and particles (>100 kilodaltons). The analyte containing aerosol droplets preferably have diameters ranging from about 0.1 μm to about 100 μm.

The flow of analyte aerosol is then ionized at block 14, preferably using one of three different ionization schemes. The first scheme ionizes the analyte aerosol by intersecting the aerosol with charged solvent droplets to form an ionized aerosol at block 16. In this embodiment at block 16, the solvent droplets may be charged by a spray ionization method such as electrospray ionization (ESI), paper spray ionization, sonic spray ionization, thermospray ionization, and other suitable spray ionization methods. In one embodiment, the charged droplets are droplets of solvent from a spray ionization solvent reservoir and the ionized aerosol is dried by evaporation of the solvent.

In another embodiment, the analyte aerosol is ionized at block 14 by passing the analyte aerosol through a photoionization source at block 18 to form the ionized aerosol. For example, the analyte aerosol may be ionized directly by the photoionization source or indirectly by photoionization of other gaseous species resulting in proton or other charge transfer to particles in the aerosol at block 18. In one embodiment, the photoionization source is an ultraviolet (UV) ionizing source. In another embodiment, the UV ionizing source is a UV transparent tubing through which the aerosol is passed, and an ultraviolet light source such as a laser, a diode laser, a flash lamp, an arc discharge source, and high intensity UV light sources ionizes the aerosol within the tube.

In another embodiment, the analyte aerosol is ionized at block 14 by passing the analyte aerosol through a cold plasma ionization source or, plasma ionization source wherein the plasma is generated with DC, AC, RF, microwave, or pulsed capacitor discharge at block 20 to form the ionized aerosol. In one embodiment, the cold plasma source at block 20 is a tube wrapped in coils connected to a high voltage RF power supply, which produces a cold plasma inside of the tubing and the analyte aerosol is bombarded by ionized molecules of the plasma resulting in ionization of the aerosol. In another embodiment, the cold plasma source is an aerosol tube and a high voltage direct current (DC) power supply configured to apply a high DC voltage within the tubing that produces a cold plasma inside of the tubing; and the aerosol is bombarded by ionized molecules of the plasma, resulting in ionization of the aerosol.

After ionization at block 14, the ionized aerosols are dried at block 22 to produce charged, gas-phase analytes for analysis at block 24. In one embodiment, the ionized aerosols are then fully dried of solvent at block 22 in the inlet of the CDMS or ion mobility instrument to form charged, gas-phase analytes for analysis at block 24.

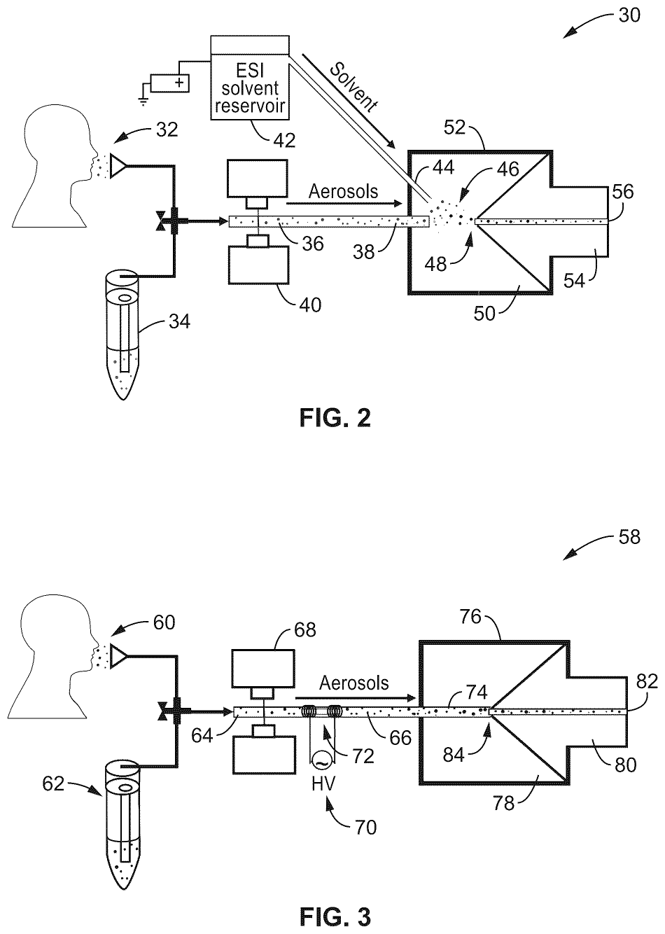

Referring now to FIG. 2, an embodiment of apparatus 30 for ionizing analyte aerosols using extractive electrospray ionization (EESI) aerosol ionization is shown schematically. In the embodiment shown in FIG. 2, the aerosol input of the apparatus comes from either the breath 32 of a subject or a nebulizer 34. The source of aerosols can be switched between breath and a nebulizer designed to generate aerosols in a size range similar to those from breath (0.1 μm to 20 μm).

The produced analyte aerosol 36 is directed through an intake tube 38 that exits into the interior 50 of an enclosure 52. The droplet size distributions of the analyte aerosol 36 flow can be verified by an aerosol size analyzer 40 that can provide high resolution and high sensitivity measurements of size distributions across the broad size range of aerosols (0.1 μm to 100+μm). This configuration allows for variations in ionization efficiency that occur as a function of aerosol size or abundance to be considered via calibration with well-characterized nebulized standards.

The apparatus 30 also has an ESI solvent reservoir 42 and duct 44 that terminates in an emitter 44 in the interior 50 of the enclosure 52 that is configured to emit charged ESI solvent droplets 46 of controllable sizes forming a plume. The flow of analyte aerosols 36 generated either by breath or nebulizer is intersected with charged droplets formed by ESI to produce ionized aerosols.

The size of the droplets 46 generated by the ESI plume depends on the capillary diameter of the emitter 44 and an optimized size may be used to maximize the ionization efficiency of the aerosol flow. Other EESI variables, such as the angles of the ESI plume and the positioning of the aerosol flow relative to the MS inlet 48, are also optimized for aerosol transmission and ionization efficiency. The ionized aerosols are then dried in the inlet 48 and drying tube 54 of the CDMS or ion mobility instrument to form gaseous ions 56 for analysis.

The apparatus 30 has an ion source capable of ionizing large (>100 kDa) biomolecules contained in aerosols and efficiently transferring them into the inlet 48 of a charge detection mass spectrometer (CDMS) or ion mobility (IMS) instrument. Extractive electrospray ionization (EESI) has been used to ionize small molecules and even intact proteins from aerosols for MS analysis. In EESI, a plume of charged droplets 46 formed from a conventional electrospray ionization (ESI) capillary is intersected with the flow of analyte-containing aerosols 36 as shown in FIG. 2. Collisions and mixing of aerosols 36 with the highly charged ESI droplets 46, followed by solvent evaporation, yields charged, gas-phase analytes 56.

Unlike previous designs aimed at small molecules, complex components aimed at removing trace volatile molecule interferences, such as heated gas denuders, are not necessary because these species will not interfere due to the much larger size of viruses. Moreover, this relatively simple design allows for aerosols to be sampled in a more direct manner, enabling a more efficient transfer of the aerosols to the MS interface.

In an alternative embodiment, an apparatus 58 utilizing cold plasma aerosol ionization is shown in FIG. 3. A flow of analyte aerosols 64 is generated either by breath 60 of a subject or with a nebulizer 62 or similar aerosol generator. The generated analyte aerosols 64 pass through tubing 66 wrapped in coils connected to a high voltage RF power supply 70, which produces a cold plasma 72 inside of the tubing 66. Aerosols 64 are bombarded by the ionized molecules of the plasma 72, resulting in ionization of the aerosols to produce ionized aerosols 74. The aerosol size distribution can be measured either before or after this ionization with an aerosol analyzer 68.

The distal end of tubing 66 is positioned to engage the MS inlet 84 and drying tube 80 within the interior 78 of the enclosure 76 to provide a direct channel. After ionization, the ionized aerosols 74 entering the inlet 84 and drying tube 80 are then fully dried of solvent in the CDMS or ion mobility instrument to form gaseous ions for analysis by the instrument.

Unlike the EESI embodiment, an aerosol ionization source based on flowing aerosols through a cold plasma does not require the turbulent intersection of gas flows that may reduce ionization and material transfer efficiencies. Cold plasma ionization is performed on aerosols 64 routed directly toward the inlet 84 of the mass spectrometer with minimal disruption and no additional reagents are required.

In one embodiment a high radiofrequency (RF) voltage is applied to coils surrounding the aerosol flow tubing 66, producing an ionizing plasma 72 between the coils and within the tube. The RF voltages and coil positioning relative to the instrument inlet are optimized for maximum ionization efficiency for a particular aerosol sample.

In another embodiment, a high direct current (DC) voltage is applied to a point within the aerosol tubing 66 to generate an ionizing plasma 72. Similar to the other aerosol ionization variants, the aerosol particle sizes generated from either a nebulizer or breath can be measured using an aerosol size analyzer 68 and the parameters optimized for efficient ionization without significant activation or dissociation of the biomolecular content contained in the aerosols. In this embodiment, as illustrated in FIG. 3, the aerosol flow 74 can be directly coupled to the MS or ion mobility inlet 84 or separated by a short distance (<1 mm) because no intersecting flows are required.

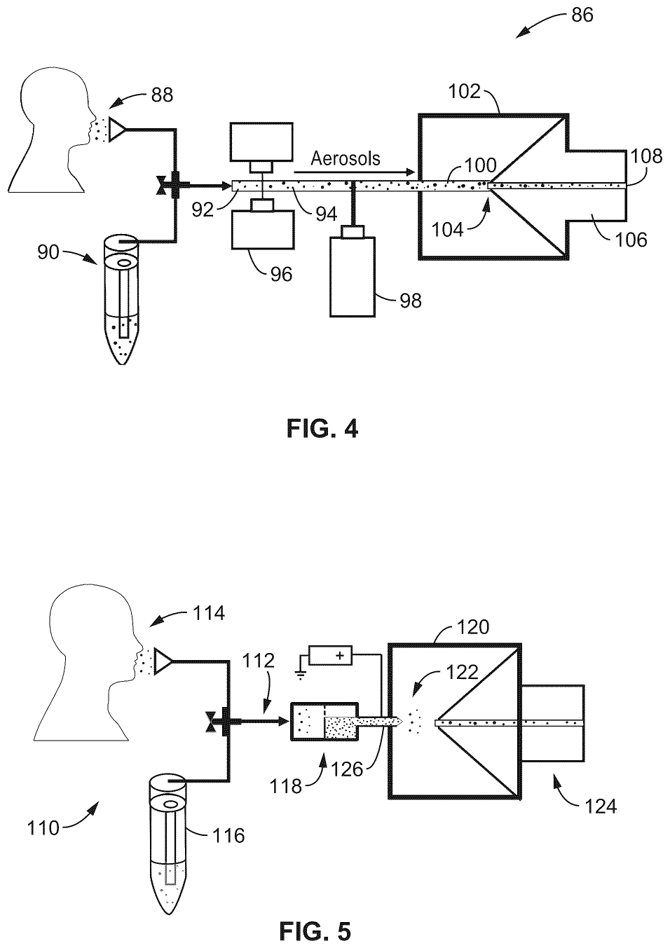

Another alternative embodiment of the apparatus 86 that uses UV photoionization aerosol ionization is shown in FIG. 4. In this embodiment, a flow of aerosols 92 is generated either by breath 88 or by nebulizer 90 or similar aerosol generation device. Aerosol size can be measured both before and after photoionization with an aerosol size analyzer 96.

The generated aerosol 90 is contained in a UV transparent tubing 94 and intersected with the beam from an ultraviolet light source 98 such as a UV diode laser, a flash lamp, an arc discharge source or some other high intensity UV light source. The distal end of the UV transparent tubing 94 is positioned to couple with or be very close to the MS inlet 104 and drying tube 106 within the interior of enclosure 102.

Aerosol particles 92 can be ionized directly or indirectly through photoionization of other gaseous species resulting in proton or other charge transfer to the aerosol particles. This results in charged aerosols 100 that are then dried in the inlet 104 of the CDMS or ion mobility instrument for mass and/or collision cross section analysis.

In this embodiment, a UV laser 98 is intersected with the aerosol flow to generate ionized aerosols via photoionization. This configuration is distinct from other methods know in the art such as atmospheric pressure chemical ionization (APCI) and atmospheric pressure photoionization (APPI), as these methods would produce highly charged droplets similar to those produced by ESI rather than directly ionizing the “dry” analyte molecules.

Here, a UV laser beam passes through a UV-transparent tubing containing the aerosol flow and generates ions via photoionization. The excess energy produced by this process is dissipated by evaporation of droplet water molecules. Even if some internal biomolecular covalent bonds are broken via UV light absorption, the large size (>1 MDa) of the biomolecules and their confinement within the aerosol droplet 92 is such that this process is unlikely to influence the mass measured in the charge detection mass spectrometer. UV power, cross section, and the volume of the aerosol flow intersected by the laser beam are variables that are optimized to maximize ionization efficiency without causing unacceptable levels of dissociation and fragmentation. Similar to other aerosol ionization embodiments, the aerosol particle sizes generated from either a nebulizer or breath can be measured and used to find optimal operational parameters. In this embodiment, the aerosol flow can be directly coupled to the MS or ion mobility inlet 104 or separated by a short distance (<1 mm) because no intersecting flows are required.

Another alternative embodiment of an apparatus 110 that condenses aerosols prior to ionization is shown in FIG. 5. In this embodiment, a flow 112 of aerosols is generated either by breath 114 or by nebulizer 116 or similar aerosol generation device. The aerosol flow 112 is directed into an aerosol condensing device 118 such as an impinger, Coriolis sampler (cyclone), impactor, or a filter. Similar to the direct aerosol ionization embodiments, the sizes of the aerosol particles sizes generated from either a nebulizer 116 or breath 114 sources can be measured with a size analyzer and used to find optimal operation parameters.

The generated condensate solution may be subsequently ionized using methods, including but not limited to, electrospray ionization (ESI), extractive ESI (ESSI), desorption ESI (DESI), cold plasma ionization, sonic spray ionization, paper spray ionization, thermospray ionization, ultraviolet (UV) photoionization, and other ionization techniques.

In the embodiment shown in FIG. 5, the generated condensate solution enters a capillary 126 configured to perform electrospray ionization of the condensate. The electrospray capillary size, potential, and geometry can be optimized to maximize ionization efficiency. The highly charged droplets 122 formed by ESI are then dried in the inlet 124 and drying tube of a charge detection mass spectrometer or ion mobility instrument to form gaseous ions for analysis.

The technology described herein may be better understood with reference to the accompanying examples, which are intended for purposes of illustration only and should not be construed as in any sense limiting the scope of the technology described herein as defined in the claims appended hereto.

### Example 1

To illustrate the functionalities and capabilities of analyte aerosol ionization methods, the EESI ionization of large biomolecules contained in aerosols was demonstrated and the process evaluated. Aerosol droplets ranging from 1 μm to 10 μm in diameter were generated from a solution of 1 μM myoglobin using a commercially available nebulizer used for administering inhaled medications (e.g., albuterol for individuals with asthma).

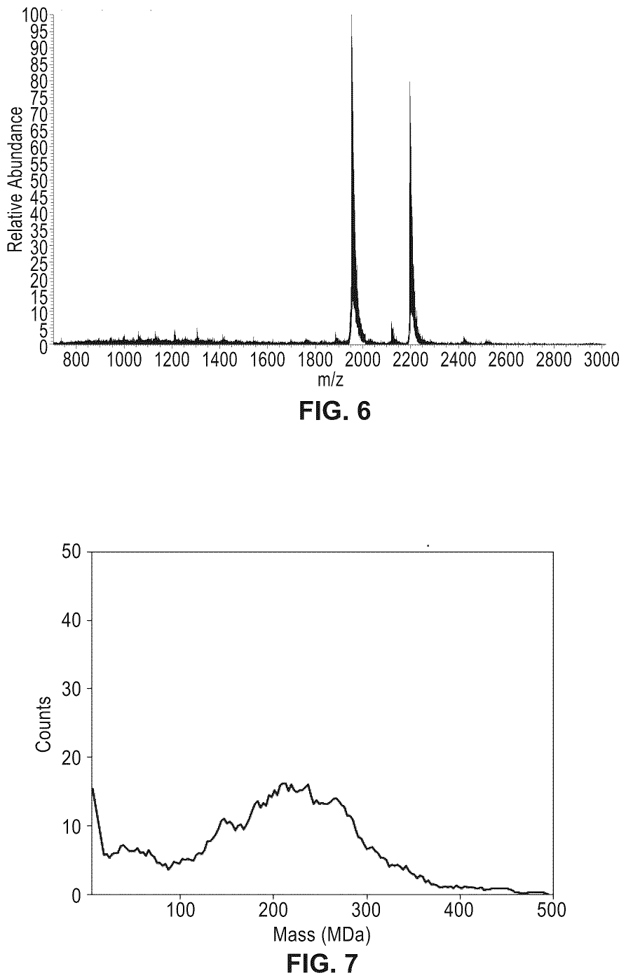

The output of the nebulizer was directed into the ESI source region of an Orbitrap mass spectrometer with an active ESI spray of an aqueous 20 mM ammonium acetate solution to perform a crude version of EESI. FIG. 6 is a plot showing the charge state distribution of holo-myoglobin observed for one orientation of the nebulizer relative to the ESI source. The native charge-state distribution indicates conditions that preserve non-covalent complexes essential for viral or bacterial analysis with this invention can be achieved using EESI. Under some orientations of the nebulizer, apo-myoglobin and denatured myoglobin species were also observed, indicating that the orientation and type of nebulization source are important in preserving these complexes. Both micro-ESI and nano-ESI spray sources were used with similar results, suggesting that achieving this aerosol ionization is not highly dependent on the flow or type of ESI aerosol droplets generated by the ion source.

### Example 2

In order to further demonstrate the EESI ionization of large biomolecule methods, droplets ranging from 1-10 μm in diameter were generated from an aqueous suspension of polystyrene nanospheres with a surface functionalized with amidine groups (0.08% by weight in solution), once again using a commercially available nebulizer used for administering inhaled medications.

The nanospheres had a nominal mean diameter of ˜95 nm. Aerosols containing nanospheres were directed into the ESI source region of a home-built CDMS instrument capable of weighing MDa-sized particles with an active ESI spray of aqueous 0.5% acetic acid and the resultant mass histogram is shown in FIG. 7. Nanospheres with masses ranging from ˜100-400 MDa were observed, indicating that EESI can be effective even for molecules with masses well into the 100+ MDa range. This is the first such demonstration of EESI for molecules in this size range.

### Example 3

The increased sensitivity of the aerosol analysis devices with the methods permits the analysis of dilute aerosols such as those produced by breath sampling devices. It is important to consider the total number of viruses or other biomolecules contained in aerosols expected to be generated from breath and the transport and ionization efficiency of those aerosols. Aerosol droplet generation from both relaxed breathing and coughing has been used to estimate the approximate number of viral copies contained per unit volume of breath. These estimates were based on SARS-CoV-2 viral loads measured from nasopharyngeal fluid and span several orders of magnitude.

Using a rough estimate of ˜500 mL for the gaseous volume of a single breath or cough, infected “high-emitters” may generate as many as 175,000 aerosol-borne viruses in a single exhalation. Some higher viral load estimates and aerosol “super-emission” behaviors (such as singing or shouting) may increase this number by an additional two orders of magnitude. In these cases, a combined ionization and transmission efficiency of ˜1% or even lower of the aerosol-borne pathogens to the detector would be sufficient to acquire a statistically robust sample of 1,000+ virus or bacteria ions in a single breath. Paired with high-throughput CDMS instrumentation or high sensitivity IMS instrumentation, combined with aerosol transport and ionization efficiency meeting this standard, highly contagious individuals should be identifiable by this technique in real time.

Further improvements in efficiency make it possible to detect progressively less contagious individuals and may even be able to detect infections at an early stage. Ionization and transmission efficiencies have not been directly measured for existing aerosol ionization systems for large biomolecules (>1 MDa), but detection limits as low as 1 femtogram/mL have been reported for EESI ionization sources applied to small molecules, indicating that highly efficient ionization and transmission can be achieved. Using the EESI and other described variants of the aerosol ionization source, it should be eminently feasible to detect and meaningfully quantify the viral and bacterial load emanated in a single breath from a contagious individual when coupled with CDMS instrumentation.

From the description herein, it will be appreciated that the present disclosure encompasses multiple implementations of the technology which include, but are not limited to, the following:

A method of ionizing an aerosol for analysis of particles in the aerosol, the method comprising obtaining a flow of aerosol; forming an ionized aerosol; and drying the ionized aerosol to yield charged, gas-phase analytes.

The method of any preceding or following implementation, wherein the aerosol is generated by a person's breath or by a nebulizer.

The method of any preceding or following implementation, wherein the ionized aerosol is formed by intersecting the aerosol with charged droplets to form an ionized aerosol.

The method of any preceding or following implementation, wherein the droplets are charged by a spray ionization method selected from the group consisting of electrospray ionization (ESI), paper spray ionization, sonic spray ionization and thermospray ionization.

The method of any preceding or following implementation wherein the charged droplets are droplets of solvent from a spray ionization solvent reservoir; and wherein the ionized aerosol is dried by evaporation of the solvent.

The method of any preceding or following implementation, wherein the ionized aerosol is formed by passing the aerosol through a plasma-based ionization source to form an ionized aerosol.

The method of any preceding or following implementation, wherein the plasma-based ionization source comprises tubing wrapped in coils connected to a high voltage RF power supply, which produces a plasma inside or vicinity of the tubing; and wherein the aerosol is bombarded by ionized molecules of the plasma, resulting in ionization of the aerosol.

The method of any preceding or following implementation wherein the plasma-based ionization source comprises an aerosol tubing and a high voltage direct current (DC) power supply configured to apply a high DC voltage within the tubing that produces a plasma inside or vicinity of the tubing; and wherein the aerosol is bombarded by ionized molecules of the plasma, resulting in ionization of the aerosol.

The method of any preceding or following implementation, wherein the ionized aerosol is formed passing the aerosol through a photoionization source to form an ionized aerosol.

The method of any preceding or following implementation, wherein the aerosol is ionized directly by the photoionization source or indirectly by photoionization of other gaseous species resulting in proton or other charge transfer to particles in the aerosol.

The method of any preceding or following implementation, wherein the photoionization source is an ultraviolet (UV) ionizing source.

The method of any preceding or following implementation, wherein the UV ionizing source comprises a UV transparent tubing through which the aerosol is passed, and an ultraviolet light source selected from the group consisting of a laser, a diode laser, a flash lamp, an arc discharge source, and high intensity UV light sources.

The method of any preceding or following implementation, wherein the aerosol is dried in an inlet of a charge detection mass spectrometer (CDMS) or an inlet of an ion mobility instrument.

An apparatus for analysis of particles in an aerosol, comprising: (a) at least one aerosol intake configured to receive an aerosol from a source; (b) an ionization tube coupled at a distal end to the intake and to an enclosure at the proximal end; (c) an ionizer adjacent to said ionization tube configured to ionize aerosol within an interior of the ionization tube; and (d) a drying tube operably coupled to the proximal end of the ionization tube.

The apparatus of any preceding or following implementation, wherein said source of aerosol comprises a nebulizer.

The apparatus of any preceding or following implementation, wherein the ionizer comprises a photoionization source or a plasma-based ionization source; wherein ionized aerosol is formed by passing the aerosol through the photoionization source or the plasma-based ionization source to form an ionized aerosol.

The apparatus of any preceding or following implementation, wherein the photoionization source is an ultraviolet light source selected from the group consisting of a laser, a diode laser, a flash lamp, an arc discharge source, and a high intensity UV light source.

The apparatus of any preceding or following implementation, wherein the plasma-based ionization source, comprises tubing wrapped in coils connected to a high voltage RF power supply, which produces a plasma inside of the tubing; and wherein the aerosol is bombarded by ionized molecules of the plasma, resulting in ionization of the aerosol.

The apparatus of any preceding or following implementation, wherein the plasma-based ionization source, comprises: a high voltage direct current (DC) power supply configured to apply a high DC voltage within the ionization tubing that produces a plasma inside of the tubing; and wherein the aerosol is bombarded by ionized molecules of the plasma, resulting in ionization of the aerosol.

The apparatus of any preceding or following implementation, wherein the drying tube is an inlet of a charge detection mass spectrometer (CDMS) or an inlet of an ion mobility instrument.

An apparatus for analysis of particles in an aerosol, comprising: (a) at least one aerosol intake configured to receive an aerosol from a source; (b) an ionization tube coupled at a distal end to the intake and to an enclosure at the proximal end; (c) a solvent reservoir; (d) a duct fluidly coupled to the solvent reservoir with an outlet configured to spray a plume of charged solvent droplets within the enclosure and positioned to intersect charged droplets with the aerosol from the ionization tube form an ionized aerosol; and (e) a drying tube within the enclosure.

The apparatus of any preceding or following implementation, further comprising an aerosol size analyzer configured to measure aerosol droplet sizes within the ionization tube.

The apparatus of any preceding or following implementation, wherein the drying tube is an inlet of a charge detection mass spectrometer (CDMS) or an inlet of an ion mobility instrument.

A method of ionizing an aerosol for analysis of particles in the aerosol, the method comprising: obtaining a flow of aerosol; condensing said aerosol to form an aerosol condensate solution; ionizing said condensate solution; and drying the ionized condensate solution to yield charged, gas-phase analytes.

The method of any preceding or following implementation, wherein the aerosol is generated by breath of a subject or by a nebulizer.

The method of any preceding or following implementation, wherein the condensation is performed by a device selected from the group consisting of aerosol impingers, impactors, Coriolis (cyclone) samplers, and filters.

The method of any preceding or following implementation, further comprising ionizing condensate using a spray ionization method selected from the group of electrospray ionization (ESI), extractive ESI (ESSI), desorption ESI (DESI), cold plasma ionization, sonic spray ionization, paper spray ionization, thermospray ionization, and ultraviolet (UV) photoionization.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, upper and lower, left and right, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A method of ionizing an aerosol for analysis of particles in the aerosol, the method comprising:
obtaining a flow of aerosol;
forming an ionized aerosol; and
drying the ionized aerosol to yield charged, gas-phase analytes.

2. The method of claim 1, wherein the aerosol is generated by a person's breath or by a nebulizer.

3. The method of claim 1, wherein the ionized aerosol is formed by intersecting the aerosol with charged droplets to form an ionized aerosol.

4. The method of claim 3, wherein the droplets are charged by a spray ionization method selected from the group consisting of electrospray ionization (ESI), paper spray ionization, sonic spray ionization and thermospray ionization.

5. The method of claim 3:
wherein the charged droplets are droplets of solvent from a spray ionization solvent reservoir; and
wherein the ionized aerosol is dried by evaporation of the solvent.

6. The method of claim 1, wherein the ionized aerosol is formed by passing the aerosol through a plasma-based ionization source to form an ionized aerosol.

7. The method of claim 6:
wherein the plasma-based ionization source comprises tubing wrapped in coils connected to a high voltage RF power supply, which produces a plasma inside or vicinity of the tubing; and
wherein the aerosol is bombarded by ionized molecules of the plasma, resulting in ionization of the aerosol.

8. The method of claim 6:
wherein the plasma-based ionization source comprises an aerosol tubing and a high voltage direct current (DC) power supply configured to apply a high DC voltage within the tubing that produces a plasma inside or vicinity of the tubing; and
wherein the aerosol is bombarded by ionized molecules of the plasma, resulting in ionization of the aerosol.

9. The method of claim 1, wherein the ionized aerosol is formed passing the aerosol through a photoionization source to form an ionized aerosol.

10. The method of claim 9, wherein the aerosol is ionized directly by the photoionization source or indirectly by photoionization of other gaseous species resulting in proton or other charge transfer to particles in the aerosol.

11. The method of claim 10, wherein the photoionization source is an ultraviolet (UV) ionizing source.

12. The method of claim 11, wherein the UV ionizing source comprises a UV transparent tubing through which the aerosol is passed, and an ultraviolet light source selected from the group consisting of a laser, a diode laser, a flash lamp, an arc discharge source, and high intensity UV light sources.

13. The method of claim 1, wherein the aerosol is dried in an inlet of a charge detection mass spectrometer (CDMS) or an inlet of an ion mobility instrument.

14. An apparatus for analysis of particles in an aerosol, comprising:
(a) at least one aerosol intake configured to receive an aerosol from a source;
(b) an ionization tube coupled at a distal end to the intake and to an enclosure at the proximal end;
(c) an ionizer adjacent to said ionization tube configured to ionize aerosol within an interior of the ionization tube; and
(d) a drying tube operably coupled to the proximal end of the ionization tube.

15. The apparatus of claim 14, wherein said source of aerosol comprises a nebulizer.

16. The apparatus of claim 15, wherein the drying tube is an inlet of a charge detection mass spectrometer (CDMS) or an inlet of an ion mobility instrument.

17. The apparatus of claim 15, further comprising an aerosol size analyzer configured to measure aerosol droplet sizes within the ionization tube.

18. The apparatus of claim 14, wherein the ionizer comprises:
a photoionization source or a plasma-based ionization source;
wherein ionized aerosol is formed by passing the aerosol through the photoionization source or the plasma-based ionization source to form an ionized aerosol.

19. The apparatus of claim 18, wherein the photoionization source is an ultraviolet light source selected from the group consisting of a laser, a diode laser, a flash lamp, an arc discharge source, and a high intensity UV light source.

20. The apparatus of claim 18, wherein the plasma-based ionization source, comprises:
tubing wrapped in coils connected to a high voltage RF power supply, which produces a plasma inside of the tubing; and
wherein the aerosol is bombarded by ionized molecules of the plasma, resulting in ionization of the aerosol.

21. The apparatus of claim 18, wherein the plasma-based ionization source, comprises:
a high voltage direct current (DC) power supply configured to apply a high DC voltage within the ionization tubing that produces a plasma inside of the tubing; and
wherein the aerosol is bombarded by ionized molecules of the plasma, resulting in ionization of the aerosol.

22. The apparatus of claim 14, wherein the drying tube is an inlet of a charge detection mass spectrometer (CDMS) or an inlet of an ion mobility instrument.

23. An apparatus for analysis of particles in an aerosol, comprising:
(a) at least one aerosol intake configured to receive an aerosol from a source;
(b) an ionization tube coupled at a distal end to the intake and to an enclosure at the proximal end;
(c) a solvent reservoir;
(d) a duct fluidly coupled to the solvent reservoir with an outlet configured to spray a plume of charged solvent droplets within the enclosure and positioned to intersect charged droplets with the aerosol from the ionization tube to form an ionized aerosol; and
(e) a drying tube with an open end in the enclosure.

24. The apparatus of claim 23, further comprising an aerosol size analyzer configured to measure aerosol droplet sizes within the ionization tube.

25. A method of ionizing an aerosol for analysis of particles in the aerosol, the method comprising:
obtaining a flow of aerosol;
condensing said aerosol to form an aerosol condensate solution;
ionizing said condensate solution; and
drying the ionized condensate solution to yield charged, gas-phase analytes.

26. The method of claim 25, wherein the aerosol is generated by breath of a subject or by a nebulizer.

27. The method of claim 25, wherein the condensation is performed by a device selected from the group consisting of aerosol impingers, impactors, Coriolis (cyclone) samplers, and filters.

28. The method of claim 25, further comprising:
ionizing condensate using a spray ionization method selected from the group of electrospray ionization (ESI), extractive ESI (ESSI), desorption ESI (DESI), cold plasma ionization, sonic spray ionization, paper spray ionization, thermospray ionization, and ultraviolet (UV) photoionization.
